(19) United States
(12) Patent Application Publication
(10) Pub. No.: US 2024/0293300 A1
(43) Pub. Date: Sep. 5, 2024

(54) SKIN PENETRATION COSMETIC MATERIAL, AND SKIN PENETRATION COSMETIC MATERIAL PRODUCTION METHOD

(71) Applicant: SHISEIDO COMPANY, LTD., Tokyo (JP)

(72) Inventors: Tomoya UCHIYAMA, Tokyo (JP); Shuting LIN, Tokyo (JP); Toru OKAMOTO, Tokyo (JP)

(21) Appl. No.: 18/572,982

(22) PCT Filed: Jul. 8, 2022
(86) PCT No.: PCT/JP2022/027175

§ 371 Date: Dec. 21, 2023

(30) Foreign Application Priority Data
Jul. 21, 2021 (JP) 2021-120922

(51) Int. Cl. A61K 8/41; A61K 8/34; A61Q 19/00

(52) U.S. Cl. CPC A61K 8/41; A61K 8/34; A61Q 19/00

(57) ABSTRACT

A skin penetration cosmetic material containing (A) a low-molecular-weight betaine, (B) a multivalent alcohol, (C) a skin condition improving ingredient, and (D) water.

### TECHNICAL FIELD

The present invention relates to a skin penetration cosmetic material, and a skin penetration cosmetic material production method.

### BACKGROUND OF THE INVENTION

In order to increase the transdermal absorbability of external preparations that are used by being applied on skin, it hitherto has been known to add an ingredient for promoting transdermal absorption of the pharmaceutical ingredients of the preparations. For example, Patent Document 1 describes use of an ion liquid made of a salt of choline and geranic acid in compositions for being locally applied on a skin surface, to improve transdermal transportation of the medical drug ingredients such as pain medications/antipyretic medications and antidepressants.

### RELATED-ART DOCUMENT

### Patent Document

Patent Document 1: Japanese Translation of PCT International Application Publication No. 2016-535781

### SUMMARY OF THE INVENTION

### Problems to be Solved by the Invention

Improvement of transdermal absorbability of effective ingredients (active ingredients) contained in external preparations is also strongly demanded in the cosmetic material field. However, the effective ingredients of cosmetic materials are ingredients such as skin lightening ingredients and anti-aging ingredients, which work to improve skin conditions. Hence, what is required of cosmetic materials is not quick permeation past skin after absorption into the skin, but penetration throughout the skin and sufficient action in the skin tissues, i.e., skin penetration. Hence, desired effects may not be obtained when techniques used in the medical drug field for promoting transdermal absorption are repurposed to cosmetic materials. Moreover, in the medical drug field, promotion of transdermal absorption may be prioritized at the cost of damage on the skin. However, cosmetic materials used by making effective ingredients penetrate the skin need to avoid adverse effects (e.g., stimulation) on the skin to the extent possible. Hence, cosmetic materials are much restricted in selection of transdermal absorption promoting ingredients.

In view of the points described above, an object of an embodiment of the present invention is to provide a skin penetration cosmetic material excellent in transdermal absorbability of a skin condition improving ingredient.

### Means for Solving the Problems

An embodiment of the present invention as a solution to the problem described above is a skin penetration cosmetic material containing (A) a low-molecular-weight betaine, (B) a multivalent alcohol, (C) a skin condition improving ingredient, and (D) water.

### Effects of the Invention

According to an embodiment of the present invention, it is possible to provide a skin penetration cosmetic material excellent in transdermal absorbability of a skin condition improving ingredient.

### BRIEF DESCRIPTION OF THE DRAWINGS

FIG. 1 is a graph indicating dissolution amounts of a skin condition improving ingredient in Example A1 and Example A2 depending on the concentration of a complex solvent in the samples.

FIG. 2 is a graph indicating dissolution amounts of a skin condition improving ingredient in Example B1 and Example B2 depending on the concentration of a complex solvent in the samples.

FIG. 3 is a graph indicating dissolution amounts of a skin condition improving ingredient in Example B1 depending on the concentration of a complex solvent in the samples.

FIG. 4 is a graph indicating changes in cumulative permeation amounts of a skin condition improving ingredient in Example 1-1 to Example 1-3 after 50% water evaporation.

FIG. 5 is a graph indicating changes in cumulative permeation amounts of a skin condition improving ingredient in Example 1-4 and Example 1-5 after 83% water evaporation.

FIG. 6 is a graph indicating changes in cumulative permeation amounts of a skin condition improving ingredient in Example 2-1 to Example 2-3.

FIG. 7 is a graph indicating differences in flux between Example 2-1 to Example 2-3.

### DETAILED DESCRIPTION OF THE INVENTION

In the present specification, “for skin penetration (or for inside-skin penetration)” means being intended for making a predetermined ingredient penetrate mainly the inside of skin. Here, “skin penetration” means not only absorption of a predetermined ingredient into the skin, but also pervasive diffusion of the predetermined ingredient inside the skin. Hence, a predetermined ingredient having a high skin penetrability includes the predetermined ingredient being easily absorbable into the skin, and also includes the predetermined ingredient being likely to be retained in the skin (also described as having an inside-skin retention property).

The “cosmetic material” may be a cosmetic product or a quasi-drug product, and is preferably in the form of a skin care product or a basic skin care product such as a lotion, a milky lotion, and a beauty essence. Moreover, the cosmetic material may be in the form of a personal care product (daily hygiene product) such as a hair conditioner, a hand cream, a body cream, and a body lotion. The texture of the cosmetic material may be such a texture as referred to as lotion, cream, emulsion, gel, and balm, and may be foam that is mixed with, or discharged while being mixed with a gas such as air. The emulsion may be an oil-in-water type, a water-in-oil type, or any other form.

The skin penetration cosmetic material (inside-skin penetration cosmetic material) according to the present embodiment has an improved transdermal absorbability, particularly, an improved skin penetrability of a skin condition improving ingredient (active ingredient, described in detail below) such as a skin lightening agent and an anti-aging agent through use of a solvent in which a low-molecular-weight betaine and a multivalent alcohol are combined. One factor that determines penetrability of a specific ingredient inside skin is distribution of the specific ingredient between a cosmetic material base and skin. According to the present embodiment, it is possible to optimize the distribution between the cosmetic material base and, particularly, the stratum corneum, which is the outermost layer of the skin. Moreover, the skin penetration cosmetic material according to the present embodiment can maintain a long-term penetration from an applied film thereof formed on the skin by application.

Some of the existing solvents used in, for example, the medical drug field cause damage on the skin, even though they may have an excellent skin penetration. As compared with this, the present embodiment does not or almost does not cause solvent-attributable damage on the skin, and can hence be applied over an area of a substantial expanse and can exert the function of the skin improving ingredient over a wide range of the skin.

### <(A) Low-Molecular-Weight Betaine>

(A) A low-molecular-weight betaine functions as a penetration promoting agent for the skin condition improving ingredient in combination with (B) a multivalent alcohol described below. (A) The low-molecular-weight betaine may have a molecular weight of 200 or less, and preferably a molecular weight of 150 or less. By using the betaine having the specified molecular weight, it is possible to reduce the possibility of skin irritation, and to alleviate or eradicate skin stimulations, i.e., uncomfortable symptoms (e.g., pain, itchiness, rash, and redness) that can be tactually or visually recognized by the user.

(A) The low-molecular-weight betaine may contain a component that forms a zwitterion in a molecule, such as a quaternary ammonium salt group, a sulfonium salt group, and a phosphonium salt group.

Moreover, as (A) the low-molecular-weight betaine, it is preferable to use a quaternary ammonium salt represented by a formula (I) below.

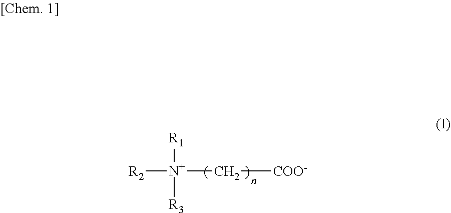

[In the formula, R₁, R₂, and R₃ each independently represent an alkyl group containing 1 or more and 6 or less carbon atoms, n represents a positive integer, and the sum total of the sum of the numbers of carbon atoms in R₁, R₂, and R₃ and n is 9 or less.]

In the formula (I), R₁ to R₃ may be a straight-chained or branched-chained alkyl group containing 1 or greater and 6 or less carbon atoms. That is, specific examples of each of R₁ to R₃ include a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group, a hexyl group, an isohexyl group, a 3-methyl pentyl group, a 2,2-dimethyl butyl group, and a 2,3-dimethyl butyl group. Here, R₁ to R₃ may be the same as or different from one another.

Specific examples of (A) the low-molecular-weight betaine include trimethyl glycine (in the formula (I), R₁ to R₃ are methyl groups, and n=1), ethyl dimethyl glycine (in the formula (I), one of R₁ to R₃ is an ethyl group and the others are methyl groups, and n=1), propyl dimethyl glycine (in the formula (I), one of R₁ to R₃ is a propyl group and the others are methyl groups, and n=1), propiobetaine (in the formula (I), R₁ to R₃ are methyl groups, and n=2), and butyrobetaine (in the formula (I), R₁ to R₃ are methyl groups, and n=3). These low-molecular-weight betaines may be used alone or in combination of two or more. Among these low-molecular-weight betaines, it is preferable to use trimethyl glycine containing fewer carbon atoms in the alkyl groups and having a high hydrophilicity.

The content of (A) the low-molecular-weight betaine relative to the whole amount of the skin penetration cosmetic material may be preferably 0.1% by mass or greater and 20% by mass or less and more preferably 1% by mass or greater and 10% by mass or less. When the content of (A) the low-molecular-weight betaine is in the range specified above, it is possible to promote dissolution of the skin condition improving ingredient and make the skin condition improving ingredient penetrate the skin at a suitable speed.

### <(B) Multivalent Alcohol>

(B) A multivalent alcohol functions as a penetration promoting agent for the skin condition improving ingredient in combination with (A) the low-molecular-weight betaine. (B) The multivalent alcohol works well to promote penetration of the skin condition improving ingredient when used in combination with (A) the low-molecular-weight betaine, and also has an advantage of low stimulation on the skin and can be used on a sensitive skin.

(B) The multivalent alcohol may be a divalent or higher and nonavalent or lower alcohol, and it is preferable that (B) the multivalent alcohol is a trivalent or higher and hexavalent or lower chained or cyclic multivalent alcohol. It is preferable that (B) the multivalent alcohol is a sugar alcohol. It is preferable that the number of carbon atoms in (B) the multivalent alcohol is 4 or greater and 12 or less.

Specific examples of (B) the multivalent alcohol include propylene glycol, 1,3-butylene glycol, pentanediol, pentylene glycol, hexanediol, hexylene glycol(2-methyl-2,4-pentanediol), isoprene glycol(3-methyl-1,3-butanediol), glycerol (glycerin), erythritol, xylitol, sorbitol, maltitol, mannitol, lactitol, and sucrose. These multivalent alcohols may be used alone or in combination or two or more. Among those mentioned above, it is preferable to use xylitol, because it can form a stable structure by forming a hydrogen bond with (A) the low-molecular-weight betaine, and tends to form a uniform, transparent phase.

The content of (B) the multivalent alcohol relative to the whole amount of the skin penetration cosmetic material may be preferably 0.1% by mass or greater 50% by mass or less and more preferably 1% by mass or greater and 10% by mass or less. When the content of (B) the multivalent alcohol is in the range specified above, the multivalent alcohol can improve transdermal absorbability of the skin condition improving ingredient together with (A) the low-molecular-weight betaine.

### <Combination of (A) Low-Molecular-Weight Betaine and (B) Multivalent Alcohol>

(A) The low-molecular-weight betaine and (B) the multivalent alcohol may be those that have intermolecular interactions between them when mixed, to result in a mixture having a significantly reduced melting point. For example, even if they are both solids at normal temperature, they may change to states that are liquid or indefinite (amorphous) at normal temperature by being mixed. (A) The low-molecular-weight betaine and (B) the multivalent alcohol according to the present embodiment can form a complex by being combined, and can constitute a Deep Eutectic Solvent (DES) or an ion Liquid (IL). A liquid solvent that is obtained by mixing a plurality of ingredients and has a melting point lower than the melting points of the ingredients before being mixed may be referred to as a complex solvent.

In the present embodiment, a complex (complex solvent) in which (A) the low-molecular-weight betaine and (B) the multivalent alcohol are combined is used. More specifically, the cosmetic material is not prepared with addition of (A) the low-molecular-weight betaine and of (B) the multivalent alcohol on a separate basis, but the cosmetic material is prepared by previously forming a complex by mixing (A) the low-molecular-weight betaine and (B) the multivalent alcohol, and then dissolving the skin condition improving ingredient in the complex solvent.

In the present embodiment, use of the complex solvent obtained by combining the ingredient (A) and the ingredient (B) optimizes the diffusion (distribution) of the skin condition improving ingredient (ingredient (C) described below) between the cosmetic material base and the skin after application of the cosmetic material. This makes it easier for the skin condition improving ingredient to be absorbed into the skin at a suitable speed. Moreover, the above formulation can make the time for which the skin condition improving ingredient is retained in the skin long. Hence, according to the present embodiment, the time for which the skin condition improving ingredient can act on the skin is long, and the skin condition improving ingredient can exert its function sufficiently.

As described above, combination of (A) the low-molecular-weight betaine and (B) the multivalent alcohol makes it possible for the skin condition improving ingredient to dissolve at a suitable concentration. Some skin condition improving ingredients are poorly soluble in the solvents commonly used in the cosmetic material field. However, with the complex solvent according to the present embodiment, it is possible to obtain a cosmetic material that contains the skin condition improving ingredient at a suitable concentration and can exert the skin condition improving function favorably. Moreover, the combination of (A) the low-molecular-weight betaine and (B) the multivalent alcohol has a high stability, particularly, a high thermal stability, and can maintain the workings of the cosmetic material for a long term. Moreover, the pH of the skin penetration cosmetic material to be obtained can be maintained in a suitable range as a cosmetic material, i.e., a range of 4.0 or higher and 10.0 or lower, and preferably 6.0 or higher and 8.0 or lower. Hence, the cosmetic material according to the present embodiment is also suitable for users who are sensitive to pH.

The ratio (M_{A}/M_{B}) of the molar quantity (M_{A}) of (A) the low-molecular-weight betaine to the molar quantity (M_{B}) of (B) the multivalent alcohol may be preferably 0.01 or greater and 10 or less, more preferably 0.1 or greater and 5 or less, and yet more preferably 0.2 or greater and 2 or less. When the ratio is in the range specified above, the ingredient (a) and the ingredient (b) are mixed well and can form a stable complex solvent. Moreover, the ratio (W_{A}/W_{B}) of the content mass (W_{A}) of (A) the low-molecular-weight betaine to the content mass (W_{B}) of (B) the multivalent alcohol may be preferably 0.005 or greater and 10 or less and more preferably 0.05 or greater and 5.0 or less.

The total amount of (A) the low-molecular-weight betaine and (B) the multivalent alcohol may be preferably 0.2% by mass or greater and 70% by mass or less and more preferably 2% by mass or greater and 20% by mass or less relative to the whole amount of the cosmetic material.

### <(C) Skin Condition Improving Ingredient>

(C) The skin condition improving ingredient is not particularly limited so long as it is an ingredient that improves a certain skin condition, and may be an ingredient that has one or more workings selected from lightening, anti-aging, antioxidation, wrinkle alleviation, macula reduction, texture improvement, firmness improvement, complexion improvement, water content improvement, hue improvement, melanin content reduction, blood circulation condition improvement, moisture retention, and cellular stimulation. It is preferable to use an ingredient mainly having a working of lightening among these workings. It is preferable that the ingredient (C) is a hydrophilic or water-soluble organic compound. (C) The skin condition improving ingredient (a medical agent that is the transdermal absorption promotion target) is not limited so long as it has any working described above, yet is preferably a hydrophilic medical agent of which the Log P value, which indicates a 1-octanol/water partition coefficient, is 3.0 or less. More preferable examples of (C) the skin condition improving ingredient include arbutin (Log P=−0.6), L-ascorbic acid (Log P=−1.6), ascorbic acid glucoside (Log P=−5.9), hydroquinone (Log P=−0.6), glutathione (Log P=−4.5), pantothenic acid (Log P=−1.1), tranexamic acid (Log P=−2), piperidine propionic acid (Log P=−2.06), Kojic acid (Log P=−0.9), L-cysteine (Log P=−2.5), ellagic acid (Log P=1.1), rucinol (Log P=2.4), resorcinol (Log P=0.8), rutin (Log P=−1.3), tryptophan (Log P=−1.1), histidine (Log P=−3.2), flavonoids such as quercetin (Log P=1.5) and quercitrin (Log P=0.9), catechin (Log P=0.4) and derivatives thereof, gallic acid (Log P=0.7) and derivatives thereof, kinetin (Log P=1), α-lipoic acid (Log P=1.7), erythorbic acid (Log P=−1.6) and derivatives thereof, thiotaurine (Log P=−0.1), urea (Log P=−1.4), nicotine (Log P=1.2) and derivatives thereof, nicotinic acid (Log P=0.4), nicotinamide (Log P=−0.4), 4-methoxysalicilic acid (Log P=2.33), hydroxyproline (Log P=−3.3), serine (Log P=−3.1), glycylglycine (Log P=−2.3), glutamic acid (Log P=−3.7), arginine (Log P=−4.2), alanine (Log P=−3), Minoxidil (Log P=1.2), D-glucosamine (Log P=−2.8), N-acetyl-D-glucosamine (Log P=−1.7), hyaluronic acid (Log P=−7.4), raffinose (Log P=−5.8), azelaic acid (Log P=1.6), γ-aminobutyric acid (Log P=−3.2), allantoin (Log P=−2.2), L-carnitine (Log P=−0.2), biotin (Log P=0.3), hydroxyethyl imidazolidinone (Log P=−1.5), pyrimidyl pyrazole compounds (Log P=3 or less) such as 2-(3,5-dimethylpyrazol-1-yl)-4,6-dimethyl pyrimidine, 2-(3,5-dimethylpyrazol-1-yl)-4,5,6-trimethyl pyrimidine, and 5-ethyl-2-(4-ethyl-3,5-dimethylpyrazol-1-yl)-4,6-dimethyl pyrimidine), and hydrochlorides thereof.

(C) The skin condition improving ingredient may be blended in the form of a salt, preferably in the form of a pharmaceutically acceptable salt, e.g., in the form of a sodium salt or a potassium salt. Moreover, (C) the skin condition improving ingredients listed as examples above may be used alone or in combination or two or more.

Log P is a coefficient indicating the polarity of a substance based on the readiness of the substance to be partitioned to water and octanol, as defined in, for example, Chemical Reviews vol 71(6), 525 (1971), and is a logarithmic value of a partition coefficient measured with 1-octanol/water (a buffer solution having pH of 7.4) obtained by a flask shaking method. The flask shaking method is a method of mixing water and 1-octanol for 24 hours or longer to saturation, and (2) adding the resulting product together with the measurement target substance into a flask and shaking them, (3) subjecting them to phase separation by centrifugation, and (4) determining the quantity of the target substance contained in each phase. Log P can also be calculated by software “EPI Suite (trademark)” (available from United States Environmental Protection Agency, https://www.epa.gov/tsca-screening-tools/download-epi-suitetm-estimation-program-interface-v411).

(C) The skin condition improving ingredient is not limited so long as it has the skin improving workings described above. It is particularly preferable to use L-ascorbic acid glucoside (Log P=−5.9), tranexamic acid (Log P=−2) and salts thereof, nicotinamide (Log P=−0.4), 4-methoxysalicylic acid (Log P=2.33) and salts thereof, glycylglycine (Log P=−2.3), hyaluronic acid (Log P=−7.4), hydroxyethyl imidazolidinone (Log P=−1.5), piperidine propionic acid (Log P=−2.06), and a pyrimidyl pyrazole compound represented by a general formula (II) below or salts thereof. Among these, it is preferable to use the pyrimidyl pyrazole compound represented by the general formula (II) below or salts thereof.

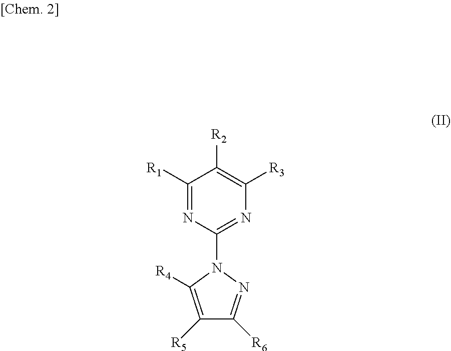

[In the formula, R₁, R₃, R₄, and R₆ are each independently an alkyl group containing from 1 through 3 carbon atoms, and R₂ and R₅ are each independently a hydrogen atom or an alkyl group containing from 1 through 3 carbon atoms.]

The ingredient may be a compound as represented above (Log P=3 or less). Specific examples include 2-(3,5-dimethylpyrazol-1-yl)-4,6-dimethyl pyrimidine and hydrochlorides thereof, 2-(3,5-dimethylpyrazol-1-yl)-4,5,6-trimethyl pyrimidine, and 5-ethyl-2-(4-ethyl-3,5-diemthylpyrazol-1-yl)-4,6-dimethyl pyrimidine. Among these, 2-(3,5-dimethylpyrazol-1-yl)-4,6-dimethyl pyrimidine hydrochloride (Log P=1.36) is preferable.

When a transdermal absorption promoting agent is blended in a cosmetic material containing the 2-(3,5-dimethylpyrazol-1-yl)-4,6-dimethyl pyrimidine hydrochloride (Log P=1.36), skin stimulation may occur depending on, for example, the overall prescription, the constitutional predisposition of the user, and conditions of use. However, when the above-described complex solvent according to the present embodiment (combination of (A) the low-molecular-weight betaine and (B) the multivalent alcohol) is used as the transdermal absorption promoting ingredient, a high skin penetrability is obtained, and stimulation during penetration throughout the skin can be reduced or eliminated.

The content of (C) the skin condition improving ingredient in the cosmetic material according to the present embodiment is preferably 0.01% by mass or greater and 10% by mass or less and may be more preferably 0.1% by mass or greater and 5% by mass or less relative to the whole amount of the cosmetic material.

The ratio (W_{C}/(W_{A}+W_{B})) of the content (W_{C}) of (C) the skin condition improving ingredient to the total of the content (W_{A}) of (A) the low-molecular-weight betaine and the content (W_{B}) of (B) the multivalent alcohol may be 0.001 or greater and 10 or less and more preferably 0.01 or greater and 3 or less on a mass or weight basis. According to a blend based on the specified ratio, (C) the skin condition improving ingredient can dissolve in the solvent favorably, and the transdermal absorbability of (C) the skin condition improving ingredient is improved and the function thereof is exerted favorably.

### <(D) Water>

(D) Water may be, for example, ion-exchanged water, purified water, and tap water. The content of the water in the cosmetic material according to the present embodiment may be preferably 10′% by mass or greater and 95% by mass or less and more preferably 30% by mass or greater and 80% by mass or less relative to the whole amount of the cosmetic material, although the content of the water depends on the texture of the cosmetic material intended to be obtained.

### <Other Ingredients>

The skin penetration cosmetic material according to the present embodiment may contain ingredients other than the ingredient (A) to the ingredient (D) described above within a range in which the effect of the present embodiment is not inhibited. The other ingredients may include a water-based ingredient other than (D) the water. More specifically, the other ingredients may include a water-soluble alcohol, a water-phase thickener, a moisturizer, a chelate agent, an antiseptic, a neutralizer, and a pigment.

The water-soluble alcohol may be a lower alcohol. Examples include ethanol, propanol, isopropanol, isobutyl alcohol, and t-butyl alcohol.

As the moisturizer, a divalent or trivalent multivalent alcohol may be used. In this case, the multivalent alcohol used as the moisturizer can be added together with either or both of the water and the water-based ingredient during preparation. It is preferable to add the moisturizer after dissolving (C) the skin condition improving ingredient in the complex solvent obtained by mixing (A) the low-molecular-weight betaine and (B) the multivalent alcohol. In this case, the multivalent alcohol as the moisturizer can be used by 0.001% by mass or greater and 50% by mass or less relative to the whole amount of the cosmetic material.

Other examples of the other ingredients includes an oil-based ingredient, a water-soluble polymer, a surfactant other than the ingredient (a), an inorganic powder, and a polymer powder. The oil-based ingredient may be, for example, a hydrocarbon oil, an ester oil, a higher fatty acid, a higher alcohol, a silicone oil, a liquid oil or fat, a solid oil or fat, a wax, a fragrance, and an oil-phase thickener.

The surfactant may be any selected from cationic surfactants, anionic surfactants, nonionic surfactants, and amphoteric surfactants.

The water-soluble polymer may be an acrylic-based polymer. More specifically, the water-soluble polymer may be, for example, a polymer containing a monomer unit derived from one or more selected from acrylic acid, methacrylic acid, acrylic acid alkyl ester, methacrylic acid alkyl ester, acrylamide, and methacrylamide, or a salt of the polymer. For example, the water-soluble polymer may be, for example, a carboxyvinyl polymer (carbomer).

### <Skin Penetration Cosmetic Material Production Method>

An embodiment of the present invention may be a skin penetration cosmetic material production method including: mixing (A) the low-molecular-weight betaine and (B) the multivalent alcohol to obtain a complex (complex solvent); adding (C) the skin condition improving ingredient to the complex; and subsequently adding (D) the water. Here, when adding (C) the skin condition improving ingredient to the complex, it is optional whether to add and mix (C) the skin condition improving ingredient without diluting the complex, i.e., without adding any other ingredient than (A) the low-molecular-weight betaine and (B) the multivalent alcohol, or to add and mix (C) the skin condition improving ingredient after diluting the complex.

An embodiment of the present invention may be a skin penetration cosmetic material produced by adding (C) the skin condition improving ingredient and subsequently adding (D) the water to a complex obtained by mixing (A) the low-molecular-weight betaine and (B) the multivalent alcohol. Here, when adding (C) the skin condition improving ingredient to the complex, it is optional whether to add and mix (C) the skin condition improving ingredient without diluting the complex, i.e., without adding any other ingredient than (A) the low-molecular-weight betaine and (B) the multivalent alcohol, or to add and mix (C) the skin condition improving ingredient after diluting the complex.

### EXAMPLES

### Test on Distribution Variation Depending on Amount of Complex Solvent (Example A1 and Example A2)

### Sample Preparation

### Example A1

To a complex solvent obtained by mixing trimethyl glycine and xylitol at a mass ratio of 3.4:6.6, ion-exchanged water was added to dilute the complex solvent to proportions of 0% by mass, 20% by mass, and 60% by mass. To the solvent diluted to each concentration, 2-(3,5-dimethylpyrazol-1-yl)-4,6-dimethyl pyrimidine hydrochloride (Log P=1.36) (a skin condition improving ingredient, which is hereinafter referred to as a compound a) was added until a precipitate of the compound a occurred. Subsequently, the resulting product was adjusted to pH of approximately 6.0 with potassium hydroxide.

### Example A2

To a complex solvent obtained by mixing dipropylene glycol and glycerin at a mass ratio of 1 ion-exchanged water was added to dilute the complex solvent to proportions of 0% by mass, 20% by mass, and 60% by mass. To each diluted solvent, the compound a was added until a precipitate of the compound a occurred. After a precipitate of the compound a occurred, the resulting product was adjusted to pH of approximately 6.0 with potassium hydroxide, as in Example A1.

<Measurement of Dissolution Amount of Compound a>

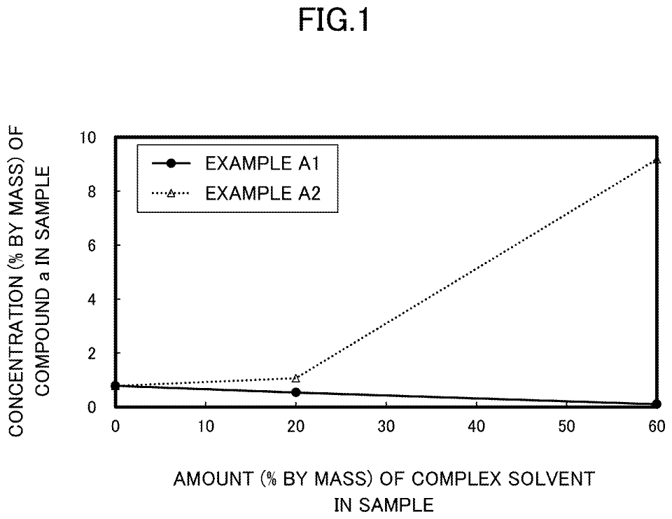

Each of the samples of Example A1 and Example A2 (a total of six water-based compositions containing the complex solvent at different proportions) was prepared in a screw tube (Maruemu Corporation) and centrifuged using a centrifuge CF 7D2 (HITACHI) at 2,300 rpm for 2 hours. Subsequently, the supernatant was removed to a CENTRIFUGE WARE (HITACHI), and ultracentrifuged using an ultracentrifuge CP100WX (HITACHI) at 65,000 rpm for 2 hours, to separate the precipitate of the compound a and recover the supernatant. The amount of the compound a in the supernatant was obtained by quantity determination using a UV detector (detection wavelength: 257 nm) of highly pressure-resistant HPLC LC-2030C (Shimadzu Corporation). FIG. 1 indicates the amount (% by mass) (saturated solubility) of the compound a vs. the amount (% by mass) of the complex solvent in the sample.

As indicated in FIG. 1, in Example A2 in which a commonly used solvent (moisturizer) was used, the concentration of the compound a (solubility in the solvent) increased as the concentration of the complex solvent increased, whereas in Example A1 in which a complex solvent made of a combination of a low-molecular-weight betaine and a multivalent alcohol was used, the solubility of the compound a in the complex solvent almost did not change but rather slightly decreased as the concentration of the solvent increased. Hence, it was inferred that use of a combination of a low-molecular-weight betaine and a multivalent alcohol would promote distribution (diffusion) of the compound a to the skin side and make it easier for the compound a to penetrate the skin, because the solubility of the compound a in the solvent decreased as the concentration of the complex solvent increased due to, for example, water evaporation.

### Test on Distribution Variation Depending on Amount of Complex Solvent (Example B1 and Example B2)

### Sample Preparation

### Example B1

To a complex solvent obtained by mixing trimethyl glycine and xylitol at a mass ratio of 3.4:6.6, ion-exchanged water was added to dilute the complex solvent to proportions of 0% by mass, 20% by mass, and 60% by mass. To the solvent diluted to each concentration, a potassium salt of 4-methoxysalicylic acid (Log P=2.33) (a skin condition improving ingredient, which is hereinafter referred to as a compound b) was added until a precipitate of the compound b occurred.

### Example B2

To a complex solvent obtained by mixing dipropylene glycol and glycerin at a mass ratio of 1:1, ion-exchanged water was added to dilute the complex solvent to proportions of 0% by mass (the same as described above), 20% by mass, and 60% by mass. To each diluted solvent, the compound b was added until a precipitate of the compound b occurred, as in Example B1.

<Measurement of Dissolution Amount of Compound b>

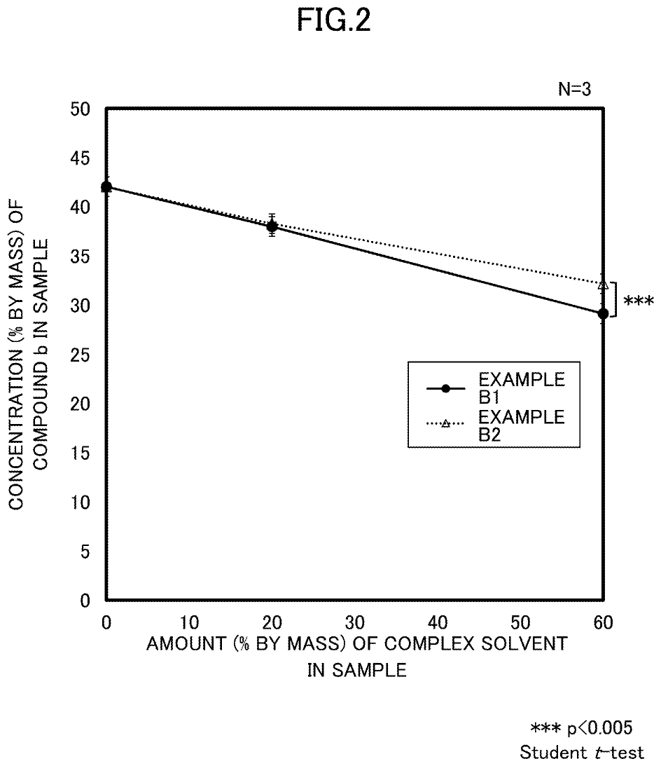

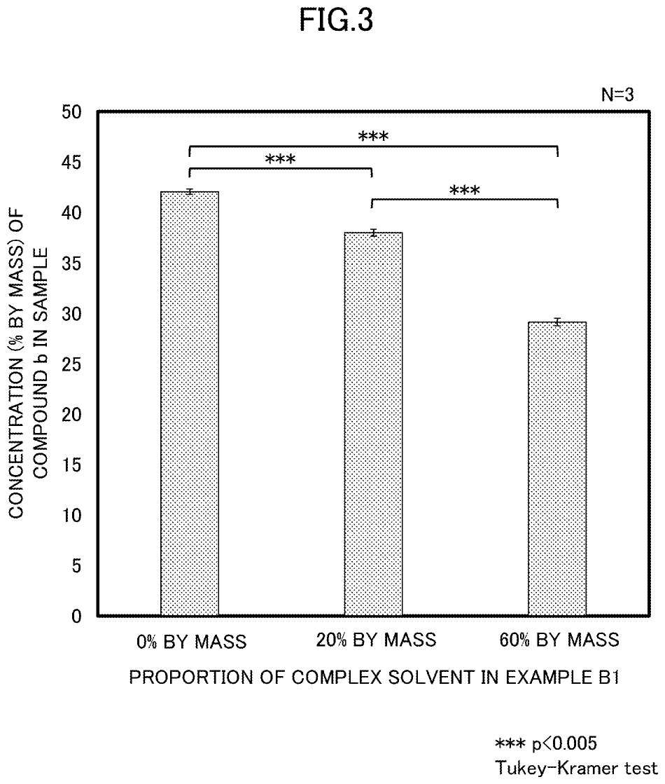

Each of the samples of Example B1 and Example B2 (a total of five water-based compositions containing the complex solvent at different proportions) was prepared in a screw tube (Maruemu Corporation), stirred using a stirrer, removed to a CENTRIFUGE WARE (HITACHI) one day later, two days later, and three days later, and ultracentrifuged using an ultracentrifuge CP100WX (HITACHI) at 50,000 rpm for 1 hour, to separate the precipitate of the compound b and recover the supernatant. The amount of the compound b in the supernatant was obtained by quantity determination using a UV detector (detection wavelength: 254 nm) of highly pressure-resistant HPLC Nexera XR (Shimadzu Corporation). FIG. 2 indicates the amount (% by mass) (saturated solubility) of the compound b vs. the amount (% by mass) of the complex solvent in the sample, regarding Example B1 and Example B2. FIG. 3 indicates the amount (% by mass) (saturated solubility) of the compound b vs. the amount (% by mass) of the complex solvent in the sample, regarding Example B1.

As indicated in FIG. 2, it was revealed that in Example B1 in which a complex solvent made of a combination of a low-molecular-weight betaine and a multivalent alcohol was used, the solubility of the compound b in the complex solvent more decreased as the concentration of the complex solvent increased, than in Example B2 in which a commonly used solvent (moisturizer) was used. Moreover, a significant difference between Example B1 and Example B2 was observed at the concentration of 60% by mass. Moreover, as indicated in FIG. 3, significant differences were observed between the respective concentrations of the combination of the low-molecular-weight betaine and the multivalent alcohol (Example B1). Hence, it was inferred that use of a combination of a low-molecular-weight betaine and a multivalent alcohol would promote distribution (diffusion) of the compound b to the skin side and make it easier for the compound b to penetrate the skin, because the solubility of the compound b in the solvent decreased as the concentration of the complex solvent increased due to, for example, water evaporation.

### =Cumulative Permeation Amount=

### <Sample Preparation I for Cumulative Permeation Amount Confirmation>

Samples of Example 1-1 and Example 1-2 were prepared in order to compare the penetration behavior of the compound a subject to volatilization of ion-exchanged water by 50% by mass from water-based compositions, which were assumed to have had a complex solvent concentration of 10% by mass and the compound a concentration of 0.3% when applied on the skin.

### Example 1-1

The compound a was added to a complex solvent obtained by mixing trimethyl glycine and xylitol at a mass ratio of 3.4:6.6. The resulting product was further diluted with ion-exchanged water, to obtain a water-based composition. Specifically, the materials were prepared such that the proportion of the total amount of trimethyl glycine and xylitol would be 20% by mass relative to the whole amount of the water-based composition and the proportion of the compound a would be 0.6% relative to the whole amount of the water-based composition, to meet the composition from which water had evaporated by 50%. Subsequently, the water-based composition was adjusted to pH of approximately 6.0 with potassium hydroxide.

### Example 1-2

The compound a was added to a complex solvent obtained by mixing dipropylere glycol and glycerin at a mass ratio of 1:1. The resulting product was further diluted with ion-exchanged water, to obtain a water-based composition. Specifically, the materials were prepared such that the proportion of the total amount of dipropylene glycol and glycerin would be 20% by mass relative to the whole amount of the water-based composition and the proportion of the compound a would be 0.6% relative to the whole amount of the water-based composition, to meet the composition from which water had evaporated by 50%. Subsequently, the water-based composition was adjusted to pH of approximately 6.0 with potassium hydroxide.

### Example 1-3

The compound a was dissolved in ion-exchanged water, to prepare a 0.6% aqueous solution corresponding to the composition from which water had evaporated by 50%. Subsequently, the aqueous solution was adjusted to pH of approximately 6.0 with potassium hydroxide, to prepare a control sample.

<Test (Cumulative Permeation Amount of Compound a)>

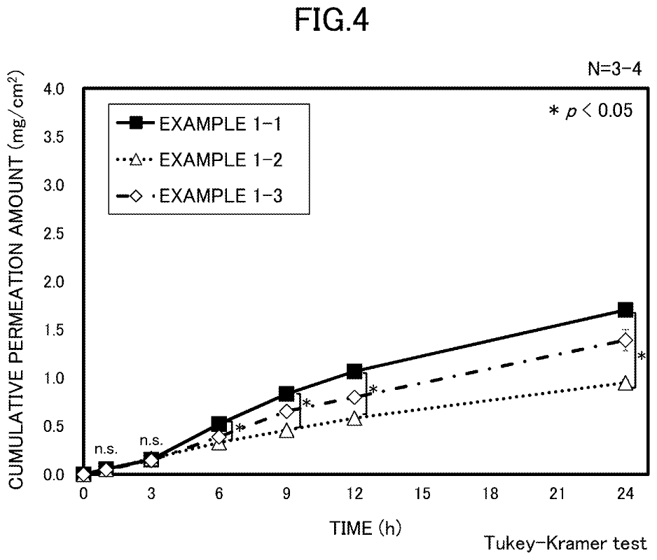

Each of the samples of Example 1-1 to Example 1-3 was applied on the stratum corneum side of an artificial skin (Strat-M (registered trademark) Membrane (Transdermal Diffusion Test Model), obtained from Merck Millipore KGaA) by 1 mL (by infinite dose), and the cumulative permeation amount was measured under an occluded condition. The artificial skin on which the sample was applied was attached on a stationary Franz cell (a “glass-made perpendicular diffusion cell” obtained from Cosmos Vid Co., Ltd., having an opening diameter of 15 mm, a film effective area of 1.77 cm², and a receptor capacity of 7.0 mL), such that the stratum corneum side would be on the donor side. The receptor was filled with a phosphate buffered saline (PBS), and the temperature of a hot water circulating portion was maintained at 35.5° C. The liquid in the receptor was sampled at the intervals of a predetermined period of time, and the quantity of the compound a was determined using a UV detector (detection wavelength: 257 nm) of highly pressure-resistant HPLC LC-2030C (Shimadzu Corporation), to obtain a cumulative permeation amount (nmol/cm²). The results are indicated in FIG. 4.

### <Sample Preparation II for Cumulative Permeation Amount Confirmation>

Samples of Example 1-4 and Example 1-5 were prepared in order to compare the penetration behavior of the compound a subject to volatilization of ion-exchanged water by 83% by mass from water-based compositions, which were assumed to have had a complex solvent concentration of 10% by mass and the compound a concentration of 0.3% when applied on the skin.

### Example 1-4

The compound a was added to a complex solvent obtained by mixing trimethyl glycine and xylitol at a mass ratio of 3.4:6.6. The resulting product was further diluted with ion-exchanged water, to obtain a water-based composition. Specifically, the materials were prepared such that the proportion of the total amount of trimethyl glycine and xylitol would be 60% by mass relative to the whole amount of the water-based composition and the proportion of the compound a would be 1.8% relative to the whole amount of the water-based composition, to meet the composition from which water had evaporated by 83%. Subsequently, the water-based composition was adjusted to pH of approximately 6.0 with potassium hydroxide.

### Example 1-5

The compound a was added to a complex solvent obtained by mixing dipropylene glycol and glycerin at a mass ratio of 1:1. The resulting product was further diluted with ion-exchanged water, to obtain a water-based composition. Specifically, the materials were prepared such that the proportion of the total amount of dipropylene glycol and glycerin would be 60% by mass relative to the whole amount of the water-based composition and the proportion of the compound a would be 1.8% relative to the whole amount of the water-based composition, to meet the composition from which water had evaporated by 83%. Subsequently, the water-based composition was adjusted to pH of approximately 6.0 with potassium hydroxide.

<Test (Cumulative Permeation Amount of Compound a)>

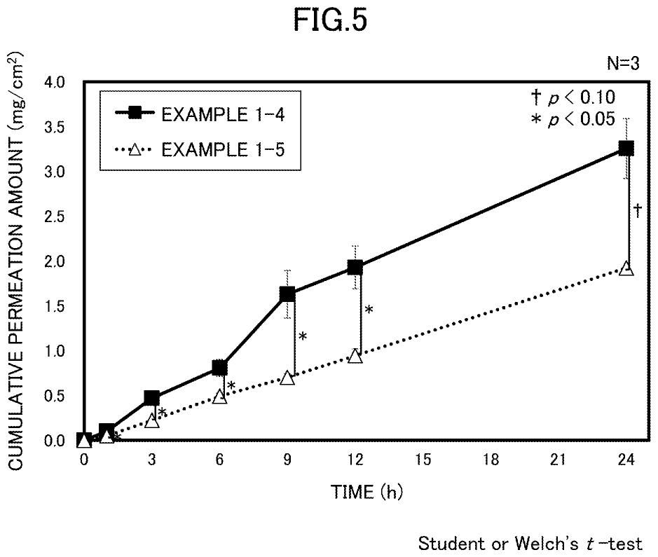

Each of the samples of Example 1-4 to Example 1-5 was applied on the stratum corneum side of an artificial skin (Strat-M (registered trademark) Membrane (Transdermal Diffusion Test Model), obtained from Merck Millipore KGaA) by 1 mL (by infinite dose), and the cumulative permeation amount was measured under an occluded condition in the same manner as in Example 1-1 and Example 1-2. The results are indicated in FIG. 5.

When a water-soluble medical agent (skin condition improving ingredient) is administered or applied by means of a water-based base, a considerable compositional change would occur due to water evaporation and affect the transdermal absorbability. It is difficult to control this change. Hence, in the examples described above, compositions having respective ingredient concentrations, which were previously assumed based on a state after a certain amount of water evaporated, were administered by an infinite dosing system, and the transdermal absorption speed (Flux) in a steady-state flow was measured. In this way, any effect by adding the composite solvent according to the present embodiment was examined.

The transdermal absorption amounts of the compound a in water-volatilized states, in which the solvent concentration became 20% by mass (water of 50% by volume had volatilized), and in which the solvent concentration became 60% by mass (water of 83% by volume had volatilized), were evaluated as in FIG. 4 and FIG. 5, respectively. According to FIG. 4, in the case where water had volatilized by 50% by volume, it was revealed that there was no difference in the transdermal absorption amount of the compound a between the sample (Example 1-2) in which DPG and glycerin were used as the solvent and the sample (Example 1-1) in which trimethyl glycine and xylitol were used as the solvent until 3 hours later. On the other hand, according to FIG. 5, in the case where water had volatilized by 83% by volume, a significant difference already occurred between the Examples (Example 1-4 and Example 1-5) after only 1 hour passed. This suggested that the absorption amount would increase through concentration of the solvent due to water volatilization.

### =Effects on Skin Penetrability=

### <Sample Preparation>

Lotions of Example 2-1 to Example 2-3 were prepared using the raw materials indicated in Table 1.

### Example 2-1

Xylitol and trimethyl glycine were mixed to obtain a complex solvent, and the compound a was dissolved in the complex solvent. Subsequently, the other ingredients were mixed.

### Example 2-2

The raw materials used were the same as in Example 2-1. However, a lotion sample of Example 2-2 was obtained, not by mixing xylitol and trimethyl glycine previously, but by adding the ingredients indicated in Table 2 into a mixing container and mixing them.

### Example 2-3

In Example 2-3, a lotion was prepared by not adding xylitol and trimethyl glycine used in Example 2-1 and Example 2-2.

TABLE 1

| | | | Ex. 2-3 |
|---|---|---|---|
| Ingredient | Ex. 2-1 | Ex. 2-2 | (control) |
| Dipropylene glycol | 7 | 7 | 7 |
| 1,3-Butylene glycol | 8 | 8 | 8 |
| Glycerin | 7 | 7 | 7 |
| Xanthane gum | 0.1 | 0.1 | 0.1 |
| Carboxyvinyl polymer | 0.25 | 0.25 | 0.25 |
| (Carbopol 981) | | | |
| Acrylates/alkyl acrylate | 0.05 | 0.05 | 0.05 |
| (C10-30) crosspolymer | | | |
| (Pemulen TR-2) | | | |
| Dimethicone (Silicone KF- | 0.5 | 0.5 | 0.5 |
| 96A-6T) | | | |
| Cetyl 2-ethyl hexanoate | 1 | 1 | 1 |
| (FineNeo-CIO) | | | |
| Any other additive | 0.723 | 0.723 | 0.723 |
| Water | balance | balance | balance |
| Compound a | 0.3 | 0.3 | 0.3 |
| Xylitol | 6.6* | 6.6 | — |
| Trimethyl glycine | 3.4** | 3.4 | — |
| Total (% by mass) | 100 | 100 | 100 |
| pH | 6.52 | 6.52 | 6.46 |

The pH of each sample was adjusted with potassium hydroxide. The pH of the lotion samples of Example 2-1 and Example 2-2 was 6.52, and the pH of the lotion sample of Example 2-3 (control) was 6.46.

<Measurement of Cumulative Permeation Amount of Compound a>

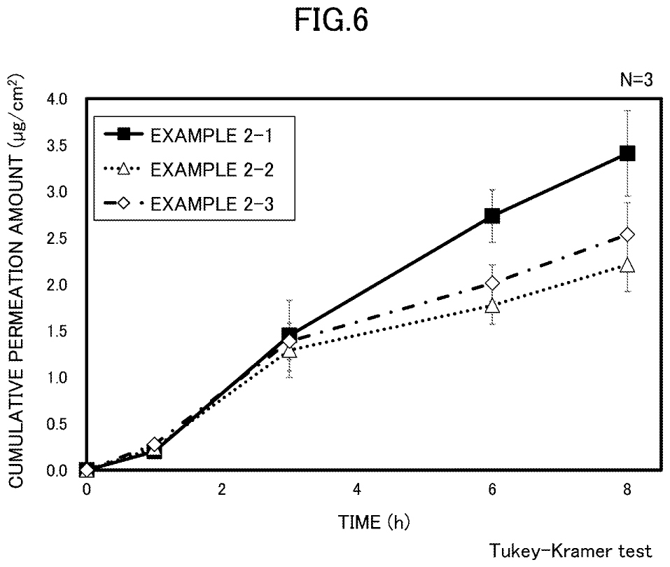

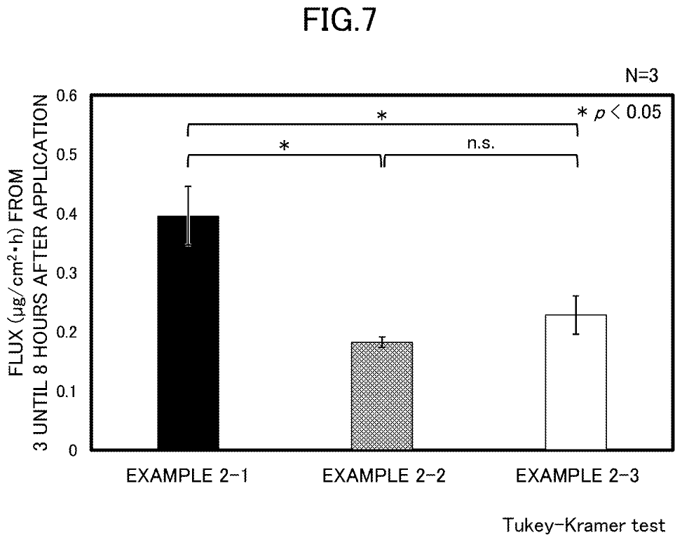

The cumulative permeation amounts (nmol/cm²) of the samples of Example 2-1 to Example 2-3 were measured in the same manner as the measurement of the cumulative permeation amounts in Example 1-1 to Example 1-3 except that the samples were applied in finite dose by 10 μL/cm² under an open condition. The results are indicated in FIG. 6. The fluxes (the slopes of the graphs in FIG. 6) from 3 hours later until 8 hours later were calculated, and compared among Examples 2-1 to Example 2-3 based on a Tukey-Kramer test. The results are indicated in FIG. 7.

The comparison among the transdermal absorption amounts of the sample (Example 2-1) in which the skin condition improving ingredient was added to the complex solvent obtained by previously mixing the low-molecular-weight betaine and the multivalent alcohol, the sample (Example 2-2) in which the low-molecular-weight betaine and the multivalent alcohol were added on a separate basis, and the sample (Example 2-3) in which the low-molecular-weight betaine and the multivalent alcohol were not added as the solvent was as indicated in FIG. 6. As indicated in FIG. 6, it was revealed that there was no flux difference among Example 2-1 to Example 2-3 until 3 hours later, but, on the other hand, that as of 3 hours later, the flux of the compound a into the skin was rapid from the sample (Example 2-1) in which the low-molecular-weight betaine and the multivalent alcohol were blended after being previously mixed. Moreover, as indicated in FIG. 7, the flux of the sample (Example 2-1) in which the complex solvent obtained by previously mixing the low-molecular-weight betaine and the multivalent alcohol was used was significantly high.

The present invention has been described based on specific embodiments and specific Examples. However, these embodiments and Examples are merely presented as examples, and the present invention is not limited by the embodiments and Examples described above. Various changes, modifications, replacements, deletions, additions, and combinations are applicable within the scope of the disclosure of the present invention.

The present application claims priority to Japanese Patent Application No. 2021-120922 filed Jul. 21, 2021, the entire contents of which are incorporated herein by reference.

1. A skin penetration cosmetic material, comprising:
(A) a low-molecular-weight betaine;
(B) a multivalent alcohol;
(C) a skin condition improving ingredient; and
(D) water.

2. The skin penetration cosmetic material according to claim 1,
wherein (B) the multivalent alcohol is one or more selected from the group consisting of glycerin, erythritol, xylitol, sorbitol, 1,3-butylene glycol, propylene glycol, pentanediol, pentylene glycol, hexanediol, glycerol, and maltitol.

3. The skin penetration cosmetic material according to claim 1,
wherein (A) the low-molecular-weight betaine is represented by a formula below, 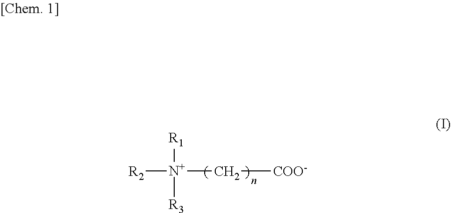
wherein in the formula, R₁, R₂, and R₃ are each independently an alkyl group containing 1 or more and 6 or less carbon atoms, n represents a positive integer, and a sum total of a sum of numbers of carbon atoms in R₁, R₂, and R₃, and n is 9 or less.

4. The skin penetration cosmetic material according to claim 1,
wherein (A) the low-molecular-weight betaine is one or more selected from the group consisting of trimethyl glycine, ethyl dimethyl glycine, propyl dimethyl glycine, propiobetaine, and butyrobetaine.

5. The skin penetration cosmetic material according to claim 1,
wherein a ratio of a molar quantity of (A) the low-molecular-weight betaine to a molar quantity of (B) the multivalent alcohol is 0.01 or greater and 10 or less.

6. The skin penetration cosmetic material according to claim 1,
wherein a total amount of a content of (A) the low-molecular-weight betaine and a content of (B) the multivalent alcohol is 0.2% by mass or greater and 70% by mass or less relative to a whole amount of the skin penetration cosmetic material.

7. The skin penetration cosmetic material according to claim 1,
wherein (A) the low-molecular-weight betaine and (B) the multivalent alcohol form a complex.

8. The skin penetration cosmetic material according to claim 1,
wherein log P of (C) the skin condition improving ingredient is 3 or less.

9. The skin penetration cosmetic material according to claim 1,
wherein (C) the skin condition improving ingredient is one or more selected from the group consisting of L-ascorbic acid glucoside, tranexamic acid, nicotinamide, 4-methoxysalicylic acid, glycylglycine, hyaluronic acid, hydroxyethyl imidazolidinone, piperidine propionic acid, and a pyrimidyl pyrazole compound represented by a general formula (II) below, 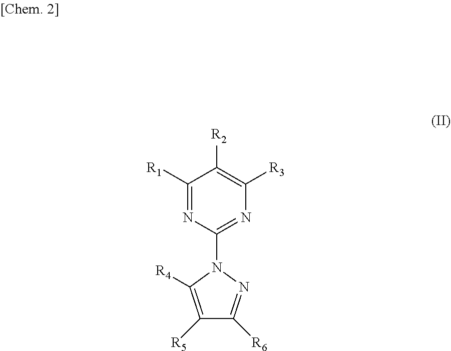
wherein in the formula, R₁, R₃, R₄, and R₆ are each independently an alkyl group containing from 1 through 3 carbon atoms, and R₂ and R₅ are each independently a hydrogen atom or an alkyl group containing from 1 through 3 carbon atoms.

10. A skin penetration cosmetic material production method, comprising:
mixing (A) a low-molecular-weight betaine and (B) a multivalent alcohol, to obtain a complex;
adding (C) a skin condition improving ingredient to the complex; and
subsequently adding (D) water.

11. A skin penetration cosmetic material produced by adding (C) a skin condition improving ingredient and subsequently adding (D) water to a complex obtained by mixing (A) a low-molecular-weight betaine and (B) a multivalent alcohol.
